(19) United States
(12) Patent Application Publication
(10) Pub. No.: US 2007/0073286 A1
(43) Pub. Date: Mar. 29, 2007

(54) Method and apparatus for an ocular procedure

(76) Inventors: Dorin Panescu, San Jose, CA (US); Chris Ikei, Baldwin Park, CA (US); Larry L. Hood, Laguna Hills, CA (US)

Correspondence Address: IRELL & MANELLA LLP, 840 NEWPORT CENTER DRIVE, SUITE 400, NEWPORT BEACH, CA, 92660, US

(21) Appl. No.: 11/242,430

(22) Filed: Sep. 29, 2005

(51) Int. Cl. A61B 18/14

(52) U.S. Cl. 606/41; 606/34

(57) ABSTRACT

A system for denaturing corneal tissue. The system includes a ground element and an electrode coupled to a power unit. The electrode is inserted into a cornea. The power unit provides power to the electrode to denature corneal tissue. The power may be at a level so that the temperature of the corneal tissue adjacent to the electrode is such that it minimizes tissue overheating. The power may be applied for a time duration up to 30 seconds at a level that does not exceed 500 mW. The surgeon may dial in an input parameter that correlates with a diopter correction in a cornea.

### BACKGROUND OF THE INVENTION

1. Field of the Invention

The present invention relates to a method and apparatus for treating ocular tissue.

2. Prior Art

Techniques for correcting vision have included reshaping the cornea of the eye. For example, myopic conditions can be corrected by cutting a number of small incisions in the corneal membrane. The incisions allow the corneal membrane to relax and increase the radius of the cornea. The incisions are typically created with either a laser or a precision knife. The procedure for creating incisions to correct myopic defects is commonly referred to as radial keratotomy and is well known in the art.

Radial keratotomy techniques generally make incisions that penetrate approximately 95% of the cornea. Penetrating the cornea to such a depth increases the risk of puncturing the Descemets membrane and the endothelium layer, and creating permanent damage to the eye. Additionally, light entering the cornea at the incision sight is refracted by the incision scar and produces a glaring effect in the visual field. The glare effect of the scar produces impaired night vision for the patient.

The techniques of radial keratotomy are only effective in correcting myopia. Radial keratotomy cannot be used to correct an eye condition such as hyperopia. Additionally, keratotomy has limited use in reducing or correcting an astigmatism. The cornea of a patient with hyperopia is relatively flat (large spherical radius). A flat cornea creates a lens system which does not correctly focus the viewed image onto the retina of the eye. Hyperopia can be corrected by reshaping the eye to decrease the spherical radius of the cornea. It has been found that hyperopia can be corrected by heating and denaturing local regions of the cornea. The denatured tissue contracts and changes the shape of the cornea and corrects the optical characteristics of the eye. The procedure of heating the corneal membrane to correct a patient's vision is commonly referred to as thermokeratoplasty.

U.S. Pat. No. 4,461,294 issued to Baron; U.S. Pat. No. 4,976,709 issued to Sand and PCT Publication WO 90/12618, all disclose thermokeratoplasty techniques which utilize a laser to heat the cornea. The energy of the laser generates localized heat within the corneal stroma through photonic absorption. The heated areas of the stroma then shrink to change the shape of the eye.

Although effective in reshaping the eye, the laser based systems of the Baron, Sand and PCT references are relatively expensive to produce, have a non-uniform thermal conduction profile, are not self limiting, are susceptible to providing too much heat to the eye, may induce astigmatism and produce excessive adjacent tissue damage, and require long term stabilization of the eye. Expensive laser systems increase the cost of the procedure and are economically impractical to gain widespread market acceptance and use.

Additionally, laser thermokeratoplasty techniques non-uniformly shrink the stroma without shrinking the Bowmans layer. Shrinking the stroma without a corresponding shrinkage of the Bowmans layer, creates a mechanical strain in the cornea. The mechanical strain may produce an undesirable reshaping of the cornea and probable regression of the visual acuity correction as the corneal lesion heals. Laser techniques may also perforate Bowmans layer and leave a leucoma within the visual field of the eye.

U.S. Pat. Nos. 4,326,529 and 4,381,007 issued to Doss et al, disclose electrodes that are used to heat large areas of the cornea to correct for myopia. The electrode is located within a sleeve that suspends the electrode tip from the surface of the eye. An isotropic saline solution is irrigated through the electrode and aspirated through a channel formed between the outer surface of the electrode and the inner surface of the sleeve. The saline solution provides an electrically conductive medium between the electrode and the corneal membrane. The current from the electrode heats the outer layers of the cornea. Heating the outer eye tissue causes the cornea to shrink into a new radial shape. The saline solution also functions as a coolant which cools the outer epithelium layer.

The saline solution of the Doss device spreads the current of the electrode over a relatively large area of the cornea. Consequently, thermokeratoplasty techniques using the Doss device are limited to reshaped corneas with relatively large and undesirable denatured areas within the visual axis of the eye. The electrode device of the Doss system is also relatively complex and cumbersome to use.

“A Technique for the Selective Heating of Corneal Stroma” Doss et al., Contact & Intraoccular Lens Medical Jrl., Vol. 6, No. 1, pp. 13-17, January-March, 1980, discusses a procedure wherein the circulating saline electrode (CSE) of the Doss patent was used to heat a pig cornea. The electrode provided 30 volts r.m.s. for 4 seconds. The results showed that the stroma was heated to 70° C. and the Bowman's membrane was heated 45° C., a temperature below the 50-55° C. required to shrink the cornea without regression.

“The Need For Prompt Prospective Investigation” McDonnell, Refractive & Corneal Surgery, Vol. 5, January/February, 1989 discusses the merits of corneal reshaping by thermokeratoplasty techniques. The article discusses a procedure wherein a stromal collagen was heated by radio frequency waves to correct for a keratoconus condition. As the article reports, the patient had an initial profound flattening of the eye followed by significant regression within weeks of the procedure.

“Regression of Effect Following Radial Thermokeratoplasty in Humans” Feldman et al., Refractive and Corneal Surgery, Vol. 5, September/October, 1989, discusses another thermokeratoplasty technique for correcting hyperopia. Feldman inserted a probe into four different locations of the cornea. The probe was heated to 600° C. and was inserted into the cornea for 0.3 seconds. Like the procedure discussed in the McDonnell article, the Feldman technique initially reduced hyperopia, but the patients had a significant regression within 9 months of the procedure.

Refractec, Inc. of Irvine Calif., the assignee of the present application, has developed a system to correct hyperopia with a thermokeratoplasty probe that is connected to a console. The probe includes a tip that is inserted into the stroma layer of a cornea. Electrical current provided by the console flows through the eye to denature the collagen tissue within the stroma. The process of inserting the probe tip and applying electrical current can be repeated in a circular pattern about the cornea. The denatured tissue will change the refractive characteristics of the eye. The procedure is taught by Refractec under the service marks CONDUCTIVE KERATOPLASTY and CK.

“Radio Frequency Conductive Keratoplasty in the Cornea: Prediction of Diopter Changes in Numerical Models” Pearce, et al., Proc. 26th Annual IEEE-EMBS Intl. Conf., San Francisco, Calif., September, 2004, discusses the relationship between collagen shrinkage and changes in dioptic power in the eye. “Phenomenological Evolution Equations for Heat-Induced Shrinkage of a Collagenous Tissue” Chen et al., IEEE Transactions on Biomedical Engineering, Vol. 45, No. 10, October, 1998, discusses how different corneal temperatures can produce different levels of collagen shrinkage for a procedure that utilizes lasers to correct vision acuities.

In a CK procedure, the surgeon applies pressure to insert the electrode into the cornea. Because the electrodes are manually inserted into the cornea, there may be variations in pressure. A higher pressure will produce less shrinkage because it is more difficult to shrink tissue under tension. It would be desirable to provide a system that minimizes variations in outcomes associated with application pressure of the electrode in a CK procedure and allows the surgeon to set energy delivery parameters in accordance with desired refractive outcomes.

### BRIEF SUMMARY OF THE INVENTION

A system that is used to denature corneal tissue. The system includes a ground element and an electrode coupled to a power unit. Power is delivered to the electrode to denature cornea tissue. The power may be limited to a level so that the temperature of the corneal tissue adjacent to the electrode is such that it minimizes tissue overheating. The power may be applied for a duration up to 30 seconds and at a level that does not exceed 500 mW per active energy-delivery electrode or transducer.

### BRIEF DESCRIPTION OF THE DRAWINGS

FIG. 1 is a perspective view of a system for denaturing corneal tissue;

FIG. 2 is an enlarged top perspective view of a probe of the system;

FIG. 3 is a bottom perspective view of the probe shown in FIG. 2;

FIG. 4 is an exploded view the probe;

FIG. 5 is a graph showing a waveform that is provided by a console of the system;

FIG. 6 is an illustration showing an electrode inserted into a cornea;

FIG. 7 is a graph showing variations in collagen shrinkage for different application pressures in CK procedures;

FIG. 8 is a graph showing variations in collagen shrinkage at lower temperatures for different application pressures for CK procedures;

FIG. 9 is a graph showing changes in refractive effects of a cornea versus the time duration of energy applied to the cornea;

FIG. 10 is a top view showing a pattern of denatured spots in a cornea.

### DETAILED DESCRIPTION

Disclosed is a system for denaturing corneal tissue. The system includes a ground element and an electrode coupled to a power unit. The electrode is inserted into a cornea. The power unit provides power to the electrode to denature corneal tissue. In order to minimize tissue overheating, the power may be at a level so that corneal tissue adjacent to the electrode does not exceed approximately 80 degrees centigrade. The power may be applied for a time duration up to 30 seconds at a level that does not exceed 500 mW per active energy-delivery electrode or transducer. The duration of energy delivery can be adjusted such that a desired refractive outcome is achieved. To improve probe stability during multi-second energy delivery, devices that carry multiple electrodes in combination with guiding markers or applanators can be used.

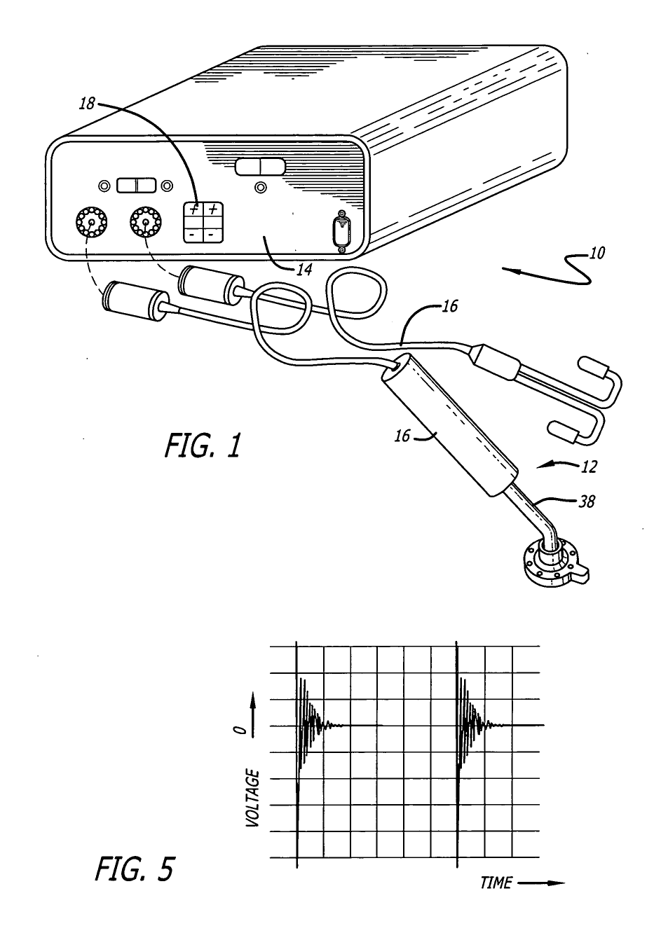

Referring to the drawings more particularly by reference numbers, FIG. 1 shows an embodiment of a system 10 that can be used to denature corneal tissue. The system 10 includes a multi-electrode probe 12 coupled to a console 14. Although a multi-electrode probe is shown in this preferred embodiment, it should be evident to the skilled in the art that the invention can be applied to single-electrode probes. The console 14 contains a power supply that can deliver electrical power to the probe 12. The system 10 may also include a return element 16 that provides a return path for current delivered by the probe 12. By way of example, the return element 16 may be a lid speculum that is used to maintain a patient's eyelids open during a medical procedure. The console 14 may have input buttons 18 that allow a surgeon to input or “dial” in settings.

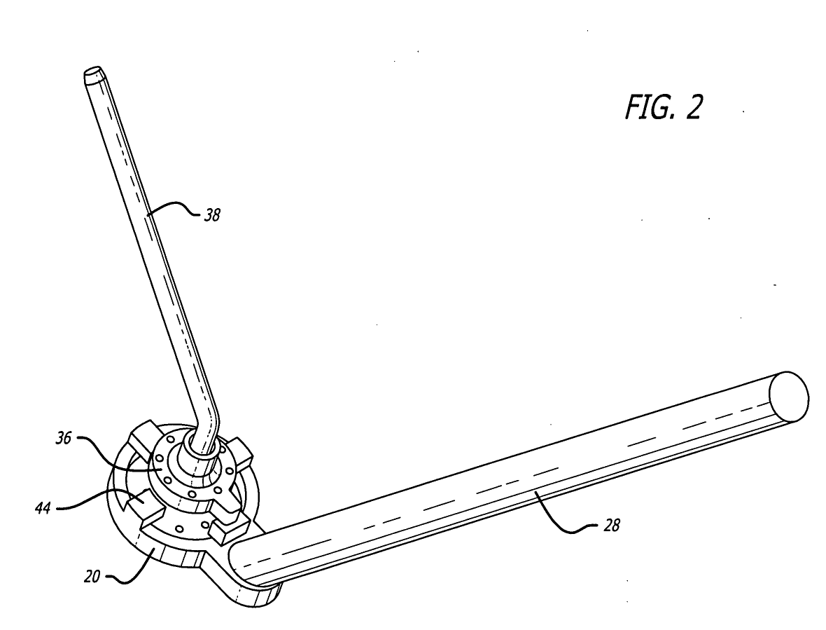

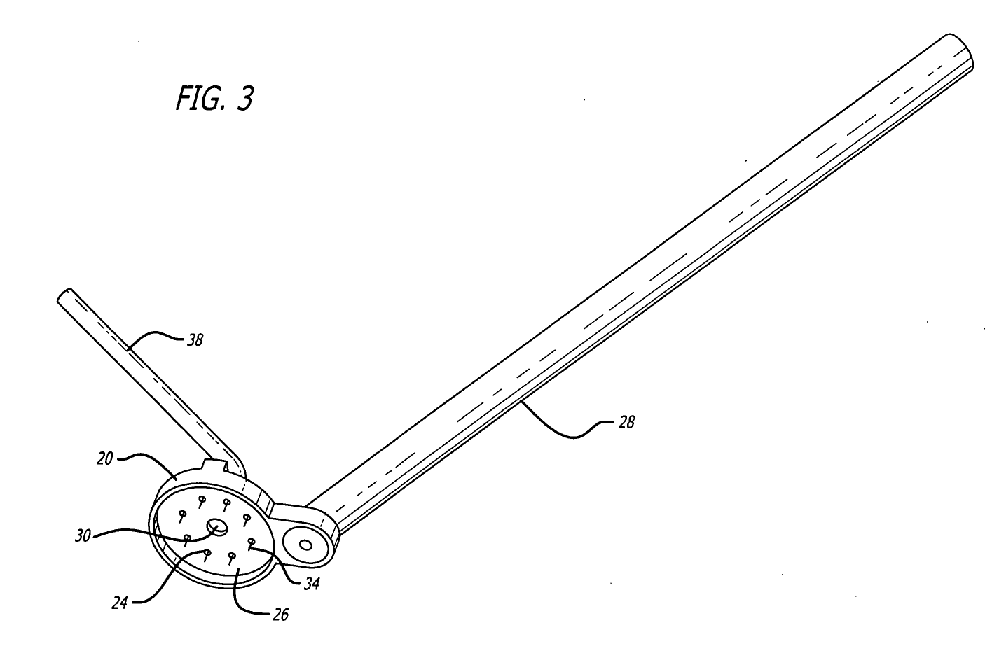

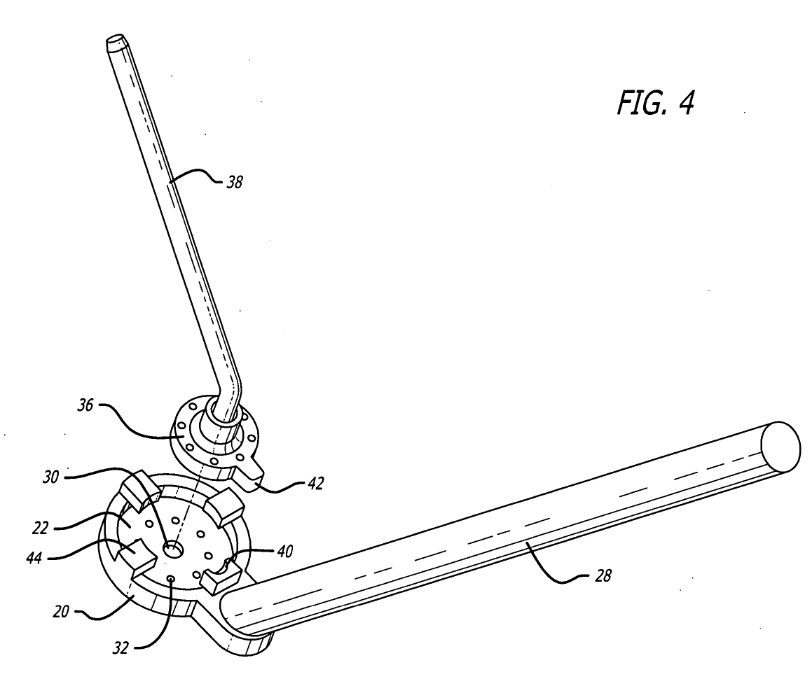

FIGS. 2-4 show an embodiment of the probe 12. The probe 12 works in combination with an applanator 20. The applanator 20 may include an upper cavity 22 and a lower cavity 24. The lower cavity 24 includes a flat bottom surface 26. The flat bottom surface 26 can be used by a surgeon to flatten a cornea. The applanator 20 includes a handle 28 that allows a surgeon to press the flat bottom surface 26 onto a cornea. The applanator 20 may include a center opening 30 that allows the surgeon to see the cornea and center the probe 12. The applanator 20 may also have a plurality of apertures 32. The apertures 32 may be equally spaced about the bottom surface 26. By way of example, the applanator 20 may have 8 apertures.

The probe 12 may further include a plurality of electrodes 34 that extend through the apertures 32. The electrodes 34 are connected to the console shown in FIG. 1. Each electrode 34 preferably has a sharp tip that allows for penetration into a cornea.

The electrodes 34 may be supported by a guide plate 36. The electrodes 34 may be connected to a connector pin 38 that is plugged into the hand piece and electronically connected to the console 14. The pin 38 may be connected to the guide plate 36 to allow for simultaneous application of energy to the electrodes 34. Alternatively, the probe 12 may include a multi-wire connector that connects the electrodes individually through the hand piece 16 to the console 14 to allow for the sequential application of energy to the electrodes 34. The guide plate 36 can be inserted into the upper cavity 22. The applanator 20 may have an alignment feature to align and prevent rotation of the guide plate 36. The alignment feature may be a groove 40 in the applanator 20 that receives a tab 42 of the guide plate 36. The upper cavity 22 may also have stops 44 that inhibit movement of the guide plate 36.

The electrodes 34 are typically constructed from a metal material. The applanator 20 and guide plate 36 may be constructed from a dielectric material such as plastic. For example, the dielectric material may be polycarbonate or other polymer, such as a polyofelin polymer. Alternatively, the applanator 20 and guide plate 36 may be constructed to include a hollow metal filled with a dielectric material.

The console 14 may provide a predetermined amount of energy to the electrodes 34, through a controlled application of power for a predetermined time duration. The console 14 may have manual controls that allow the user to select treatment parameters such as the power and time duration. The console 14 can also be constructed to provide an automated operation. The console 14 may have monitors and feedback systems for measuring physiologic tissue parameters such as tissue impedance, tissue temperature and other parameters, and adjust the output parameters of the radio frequency amplifier to accomplish the desired results.

In one embodiment, the console 14 provides voltage limiting to prevent arcing. To protect the patient from overvoltage or overpower, the console 14 may have an upper voltage limit and/or upper power limit monitor which terminates power to the probe when the output voltage or power of the unit exceeds a predetermined value.

The console 14 may also contain monitor and alarm circuits which monitors physiologic tissue parameters such as the resistance or impedance of the load and provides adjustments and/or an alarm when the resistance/impedance value exceeds predefined limits. The adjustment feature may change the voltage, current, power and/or other parameters delivered by the console such that the desired physiological change to the cornea is achieved. The alarm may provide either an audio and/or visual indication to the user that the resistance/impedance value has exceeded the predefined tolerance limits. Additionally, the unit may contain a ground fault or loss or return path indicator, and/or a tissue temperature monitor. The front panel of the console 14 typically contains meters and displays that provide an indication of the power, frequency, etc., of the power delivered to the probe.

The console 14 may deliver a radiofrequency (RF) power output in a frequency range of 100 KHz-30 MHz. In the preferred embodiment, power is provided to the probe at a frequency in the range of 350 KHz. The time duration of each application of power to a particular location of tissue can be up to several seconds.

If the system incorporates temperature sensors, the console 14 may control the power such that the target tissue temperature is maintained to no more than approximately 80° C. The temperature sensors can be carried by the probe 12, incorporated into the electrodes 34, or attached within proximity to the electrodes 34.

If the system includes an impedance monitor, the power could be adjusted so that the target tissue impedance, assuming a probe 12 with a tip of length 460 um and diameter of 90 um, decreases by approximately 50% from an initial value that is expected to range between 1100 to 1800 ohm. If two or more electrodes are energized in parallel, the initial impedance values may be less than 1000 ohm. For example, if probe 12 carries 8 electrodes 34 that are energized simultaneously, then the initial overall impedance value is expected to be in the range of 1/8 of the range above, namely 137 to 225 ohms. The console 14 could regulate the power down if, after an initial descent, the impedance begins to increase. Controls can be incorporated to terminate RF delivery if the impedance changes by a significant percentage from the baseline. Alternatively, or additionally, the console 14 could modulate the duration of RF delivery such that delivery is terminated only when the impedance exceeds a preset percentage or amount from a baseline value, unless an upper time limit is exceeded. Other time-modulation techniques, such as monitoring the derivative of the impedance, could be employed. Time-modulation could be based on physiologic parameters other than tissue impedance (e.g. tissue water content, chemical composition, etc.)

FIG. 5 shows a typical voltage waveform that is delivered by the probe 12 to the skin. Each pulse of energy delivered by the probe 12 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

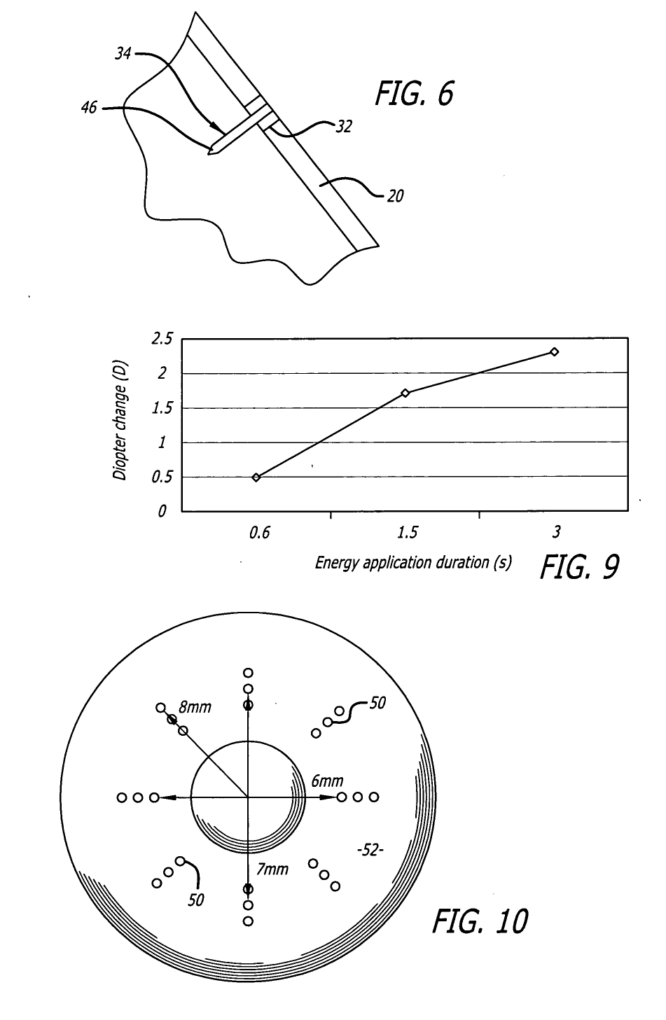

FIG. 6 shows one of the electrodes 34 inserted into a cornea. The pointed tip 46 of the electrode 34 assists in the penetration of the cornea. The applanator 20 can flatten the cornea so that the electrode 34 can be inserted in a direction that is essentially perpendicular to the outer cornea surface. The tips are typically inserted until the guide plate 36 becomes fully seated within the upper cavity 22 of the applanator 20.

Each electrode 34 should have a length that insures sufficient penetration into the stroma layer of the cornea. By way of example, the electrodes 34 may each have a length between 300 to 800 microns. The diameter of the each electrode 34 should be sufficient to provide the desired amount of energy but be small enough to not leave unsightly incision wounds. In one embodiment, the diameter of each electrode 34 is 90 microns. The electrodes 34 could carry, have embedded in it, or otherwise attached to it, specialized sensors (not shown), such as temperature sensors (e.g. thermocouples, thermistors, etc.), pressure sensors, etc. Although specific lengths and diameters have been disclosed, it is to be understood that the tip may have different lengths and diameters.

In operation, a surgeon pushes the applanator 20 onto a cornea. The applanator 20 flattens the cornea. The surgeon can then load the guide plate 36 into the upper cavity of the applanator. Loading the guide plate 36 inserts the electrodes 34 into the cornea. The surgeon then activates the power unit to deliver energy to the electrodes. The energy flows from the electrodes 34, through the cornea and to the return element. The current generates heat that denatures the collagen tissue of the stroma. The electrodes 34 can deliver the current either sequentially or simultaneously to the cornea.

The console 14 preferably delivers energy at a level and time duration so that the corneal tissue adjacent to the electrode shrinks in a structured way that provides longer lasting effects. To achieve that, it is preferred to have the corneal tissue temperature not exceed approximately 80 degrees centigrade. It is has been found that applying power for a time duration up to 30 seconds at a level that does not exceed 500 mW per active energy-delivery electrode better increases the chances of structured collagen shrinkage. This power application is to be distinguished from existing CK applications wherein the power level is around 1 W and the time duration generally does not exceed 1 second.

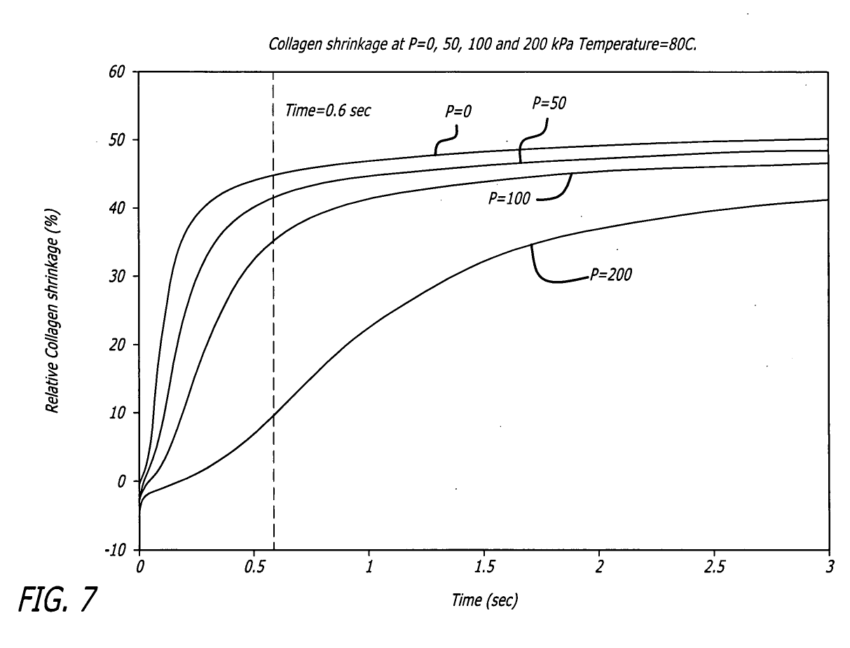

As shown in FIG. 7, assuming a tissue temperature of 80 C, for short energy-delivery durations variations in the collagen fiber tension—as may result from variations in probe application pressure—could yield significant variations in the amount of collagen shrinkage. However, if the energy delivery is extended to several seconds then the resulting collagen shrinkage is less sensitive to changes in the fiber tension. This result is confirmed by the shrinkage-time curves shown in FIG. 7 which shows the curves converging with increasing time.

As presented by Pearce et. al., the amount of corneal collagen shrinkage is one of the main factors that determine refractive outcomes. The Pearce et. al. collagen shrinkage vs. diopter change numerical model predicts almost linear dependency between the change in diopters from the baseline diopter number and the amount of corneal collagen fiber shrinkage. Therefore, FIG. 7 infers that more predictable effects might be obtained if the energy-delivery duration is extended to several seconds.

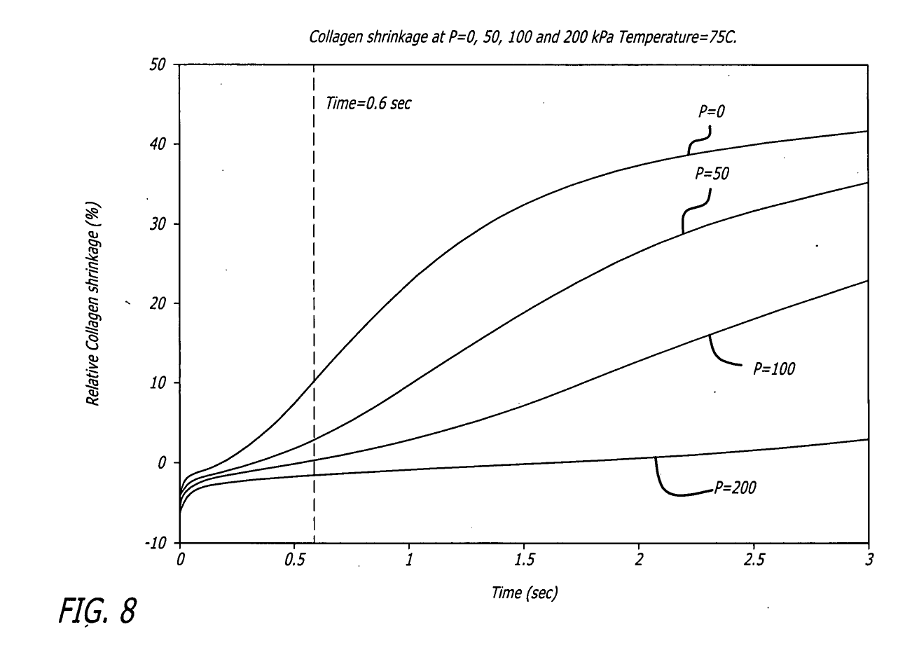

FIG. 8 shows that a variation in collagen shrinkage caused by variations in fiber tension could be even higher in corneal tissue exposed to lower temperatures (e.g. 75 C). However, the curves shown in FIG. 8 for a lower temperature also converge over time. Lower corneal tissue temperatures might be desirable in order to avoid tissue overheating associated with increased energy-delivery durations. For this reason, increased energy-delivery durations should be offset by decreased levels of power applied to the tissue.

FIG. 8 also illustrates another potential advantage of energy delivery at lower power settings for increased durations. As seen, the collagen-shrinkage curves that correspond to fiber tensions within normal range (P=0-100 kPa) display a quasi-linear dependence on duration. For example, for a fiber tension of P=50 kPa, a duration of 1.5 s produces about 20% relative collagen fiber shrinkage. With the same fiber tension, a duration of 3 s produces about 40% relative collagen fiber shrinkage. Consequently, given that the amount of collagen shrinkage can be directly linked to the amount of refractive effect (per Pearce et. al. model), this phenomenon would allow the surgeon to ‘dial’ a duration setting that would indirectly provide the desired amount of diopter correction. The model presented by Pearce et el. predicts an approximate 60% sensitivity of diopter change to relative collagen shrinkage. Based on this assumption, FIG. 9 shows a curve that can be employed by a surgeon to set the energy application duration such that a desired refractive effect is achieved. This curve is representative of an 8-spot at 8-mm diameter treatment pattern.

The surgeon can use such a graph to dial in a diopter correction for the patient. For example, to obtain a 0.5-diopter correction with an 8-spot/8-mm treatment, the surgeon may input 0.6 second through the control panel of the console. Likewise, the surgeon can dial in a 1.5-second energy duration to obtain approximately a 1.7-diopter correction. To use this embodiment of the present invention, it is preferable the user employ a device that would make the application pressure be more uniform among treatment spots. The applanator 20 with probe 12 could be used for this purpose. Alternatively, other guide devices, such as the embodiments disclosed in application Ser. No. ______ filed on Sep. 8, 2005, by the assignee of this application, which is hereby incorporated by reference, could be employed to make the spot-to-spot probe application pressure mode uniform.

The console 14 could have a built-in algorithm or look-up table that would allow the operator to select the appropriate power and time to achieve the desired diopter correction using a given surgical probe. By way of example, the algorithm or look-up table could be the same or similar to the graph shown in FIG. 9. Alternatively, the user/operator could be provided with a nomogram that can be used to manually adjust the power and time settings. To secure effective treatment, console 14 could deliver the desired power settings based on a set curve, rather than just a set point. The set curve could, for example, consist of a higher power level in the first 100-200 ms of energy application followed by lower power levels for the remaining delivery interval. Alternatively, a power set curve with higher power levels in the last 100-200 ms of the energy-delivery interval could be used. These combinations of higher/lower set values would have the effect of producing controlled tissue desiccation just in certain desired areas (e.g. around the electrode or at the border of the affected stromal zone).

FIG. 10 shows a pattern of denatured areas 50 that have been found to correct hyperopic or presbyopic conditions. A circle of 8, 16, or 24 denatured areas 50 are created about the center of the cornea, outside the visual axis portion 52 of the eye. The visual axis has a nominal diameter of approximately 5 millimeters. It has been found that 16 denatured areas provide the most corneal shrinkage and less post-op astigmatism effects from the procedure. The circles of denatured areas typically have a diameter between 6-8 mm, with a preferred diameter of approximately 7 mm. If the first circle does not correct the eye deficiency, the same pattern may be repeated, or another pattern of 8 denatured areas may be created within a circle having a diameter of approximately 6.0-6.5 mm either in line or overlapping. Given that several treatment spots are usually required during a procedure, a multi-electrode probe (such as probe 12 shown in FIGS. 2-4) would be desired because it shortens the overall procedure time if the electrodes are activated simultaneously. Additionally, in conjunction with applanator 20, or an alike guiding device, the multi-electrode probe could provide better stability in the hands of a surgeon.

The exact diameter of the pattern may vary from patient to patient, it being understood that the denatured spots should preferably be formed in the non-visionary portion 52 of the eye. Although a circular pattern is shown, it is to be understood that the denatured areas may be located in any location and in any pattern. In addition to correcting for hyperopia, the present invention may be used to correct astigmatic conditions. For correcting astigmatic conditions, the denatured areas are typically created at the end of the astigmatic flat axis. The present invention may also be used to correct procedures that have overcorrected for a myopic condition.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC) and microwave, ultrasound or light that can be transferred into the skin tissue through the probe. For example, although a multi-electrode probe was described as part of the preferred embodiment, single-electrode probes can be used as well.

By way of example, the console can be modified to supply energy in the microwave frequency range or the ultrasonic frequency range. By way of example, the probe may have a helical microwave antenna with a diameter suitable for delivery into the tissue. The delivery of microwave energy could be achieved with or without tissue penetration, depending on the design of the antenna. The system may modulate the microwave energy in response to changes in the characteristic impedance.

What is claimed is:

1. A system that is used to denature corneal tissue, comprising:
an electrode;
a return element; and,
a power unit that delivers energy no greater than 500 mW to said electrode for a time up to 30 seconds, to denature tissue of a cornea.

2. The system of claim 1, further comprising an applanator used to apply pressure to the cornea.

3. The system of claim 1, wherein said energy is provided as a highly damped repetitive waveform.

4. The system of claim 1, further comprising another electrode coupled to said power unit.

5. The system of claim 1, wherein a power of said energy varies during the delivery of said energy.

6. The system of claim 1, wherein said electrode has a tip for insertion into a cornea.

7. A system that is used to denature corneal tissue, comprising:
an electrode;
a return element; and,
power means for delivering energy no greater than 500 mW to said electrode for a time up to 30 seconds, to denature tissue of a cornea.

8. The system of claim 7, further comprising an applanator used to apply pressure to the cornea.

9. The system of claim 7, wherein said energy is provided as a highly damped repetitive waveform.

10. The system of claim 7, wherein a power of said energy varies during the delivery of said energy.

11. The system of claim 7, further comprising another electrode coupled to said power unit.

12. The system of claim 7, wherein said electrode has a tip for insertion into a cornea.

13. A system that is used to denature corneal tissue, comprising:
an electrode;
a return element; and,
a power unit that delivers energy to denature tissue of a cornea at a level and duration so that a temperature of a corneal tissue in contact with said electrode minimizes tissue overheating during the application of the energy.

14. The system of claim 13, further comprising an applanator used to apply pressure to the cornea.

15. The system of claim 13, wherein said energy is provided as a highly damped repetitive waveform.

16. The system of claim 13, wherein a power of said energy varies during the delivery of said energy.

17. The system of claim 13, further comprising another electrode coupled to said power unit.

18. The system of claim 13, wherein said electrode has a tip for insertion into a cornea.

19. A system that is used to denature corneal tissue, comprising:
an electrode;
a return element; and,
power means for delivering energy to denature tissue of a cornea at a level and duration so that a temperature of a corneal tissue in contact with said electrode minimizes tissue overheating during the application of the energy.

20. The system of claim 19, further comprising an applanator used to apply pressure to the cornea.

21. The system of claim 19, wherein said energy is provided as a highly damped repetitive waveform.

22. The system of claim 19, wherein a power of said energy varies during the delivery of said energy.

23. The system of claim 19, further comprising another electrode coupled to said power unit.

24. The system of claim 19, wherein said electrode has a tip for insertion into a cornea.

25. A method for denaturing a cornea, comprising:
providing a return path for a patient;
inserting an electrode into the cornea; and,
delivering energy to the cornea through the electrode at a level no greater than 500 mW for a time up to 30 seconds to denature tissue of a cornea.

26. The method of claim 25, wherein energy is delivered to the cornea in a pattern about the cornea.

27. The method of claim 26, wherein the pattern is created with a plurality of electrodes.

28. The method of claim 25, wherein a power of the energy varies during the delivery of the energy.

29. The method of claim 25, wherein the pattern is established by apertures of a guide plate that support the electrodes.

30. A method for denaturing a cornea, comprising:
providing a return path for a patient;
inserting an electrode into the cornea; and,
delivering energy to the cornea through the electrode to denature corneal tissue at a level and duration so that a temperature of a corneal tissue in contact with the electrode minimizes tissue overheating during the application of the energy.

31. The method of claim 30, wherein energy is delivered to the cornea in a pattern about the cornea.

32. The method of claim 31, wherein the pattern is created with a plurality of electrodes.

33. The method of claim 30, wherein a power of the energy varies during the delivery of the energy.

34. The method of claim 32, wherein the pattern is established by apertures of a guide plate that support the electrodes.

35. A system that is used to denature corneal tissue, comprising:
an electrode;
a return element; and,
a console connected to said electrode and said return element, said console having an input that allows a surgeon to input a parameter that correlates to a diopter correction of a cornea and delivers energy to said electrode.

36. The system of claim 35, further comprising an applanator used to apply pressure to the cornea.

37. The system of claim 35, wherein said energy is provided as a highly damped repetitive waveform.

38. The system of claim 35, wherein the parameter is a time duration of power applied to the cornea.

39. The system of claim 35, wherein a power of said energy varies during the delivery of said energy.

40. The system of claim 35, wherein said electrode has a tip for insertion into a cornea.

41. A system that is used to denature corneal tissue, comprising:
an electrode;
a return element; and,
console means for allowing a surgeon to input a parameter that correlates to a diopter correction of a cornea and delivering energy to said electrode.

42. The system of claim 41, further comprising an applanator used to apply pressure to the cornea.

43. The system of claim 41, wherein said energy is provided as a highly damped repetitive waveform.

44. The system of claim 41, wherein the parameter is a time duration of power applied to the cornea.

45. The system of claim 41, wherein a power of said energy varies during the delivery of said energy.

46. The system of claim 41, wherein said electrode has a tip for insertion into a cornea.

47. A method for denaturing a cornea, comprising:
inputting a parameter into a console, the parameter correlates to a diopter correction;
inserting an electrode into the cornea; and,
delivering energy to the cornea through the electrode to denature corneal tissue and achieve the diopter correction.

48. The method of claim 47, wherein energy is delivered to the cornea in a pattern about the cornea.

49. The method of claim 48, wherein the pattern is created with a plurality of electrodes.

50. The method of claim 47, wherein a power of the energy varies during the delivery of the energy.

51. The method of claim 48, wherein the pattern is established by apertures of a guide plate that support the electrodes.

52. The method of claim 47, wherein the parameter is a time duration of power applied to the cornea.
